(19) United States
(12) Patent Application Publication
(10) Pub. No.: US 2025/0017918 A1
(43) Pub. Date: Jan. 16, 2025

(54) ORAL SUSPENSIONS COMPRISING TEMOZOLOMIDE OR LENALIDOMIDE

(71) Applicant: SHORLA PHARMA LTD, Co. Tipperary (IE)

(72) Inventors: Sharon CUNNINGHAM, Co. Tipperary (IE); Orlaith RYAN, Co. Tipperary (IE); Denis DELANEY, Co. Tipperary (IE); Maurice CLANCY, Co. Tipperary (IE); Rod BAKER, Oxon (GB)

(21) Appl. No.: 18/764,489

(22) Filed: Jul. 5, 2024

Related U.S. Application Data
(60) Provisional application No. 63/525,276, filed on Jul. 6, 2023.

(51) Int. Cl. A61K 31/495; A61K 9/00; A61K 9/10; A61K 31/454; A61K 47/02; A61K 47/10; A61K 47/14; A61K 47/26

(52) U.S. Cl. CPC A61K 31/495; A61K 9/0053; A61K 9/10; A61K 31/454; A61K 47/02; A61K 47/10; A61K 47/14; A61K 47/26

(57) ABSTRACT

The present disclosure provides a composition comprising temozolomide or lenalidomide, a medium chain triglyceride such as miglyol, silicon dioxide and a surfactant, wherein the composition optionally comprises an antioxidant and/or a flavoring agent, and wherein the temozolomide composition is a non-aqueous, preservative free suspension. Also provided is a method for treating cancer in a subject, such as a child or a subject suffering dysphagia and/or has difficulty swallowing.

### CROSS-REFERENCE TO RELATED APPLICATIONS

This application claims the benefit and priority to U.S. Ser. No. 63/525,276, filed on Jul. 6, 2023. The entire disclosure of the application identified in this paragraph is incorporated herein by reference.

### FIELD

The present disclosure relates to compositions for oral administration comprising temozolomide or lenalidomide and excipients, and methods for using the same for treating a disease in a subject.

### BACKGROUND

Temozolomide (3,4-dihydro-3-methyl-4-oxoimidazo[5,1-d]-as-tetrazine-8-carboxamide) is an alkylating agent used as an oral chemotherapeutic drug. Temozolomide is indicated for treatment of glioblastoma multiforme (GBM) and refractory anaplastic astrocytoma. Temozolomide is unstable in aqueous solutions, which leads to the generation of impurities and/or temozolomide degradation products following storage. The instability renders ready-to-use liquid dosage forms difficult to store.

Temozolomide is currently administered as 5-250 mg capsules or reconstituted from 10 mg powder for IV infusion. Although temozolomide is not indicated for pediatric use, it is administered to children in off-label use.

Lenalidomide (1-oxo-2-(2,6-dioxopiperidin-3-yl)-4-aminoisoindoline) is an immunomodulatory drug known as a cereblon E3 ligase modulator. It is used to treat multiple myeloma, mantle cell lymphoma, follicular lymphoma, marginal zone lymphoma and myelodysplastic syndromes (MDS). Lenalidomide is unstable in aqueous solutions, which leads to the generation of impurities and/or lenalidomide degradation products following storage. The instability renders ready-to-use liquid dosage forms difficult to store. Lenalidomide is currently administered as 2.5-25 mg capsules.

U.S. Pat. No. 5,260,291 first reported temozolomide, polymorphs thereof, and compositions thereof. The reported compositions further comprise dimethyl sulphoxide and arachis oil.

U.S. Pat. No. 6,251,886 reports methods for treating neoplastic meningitis cancer with a microcrystalline suspension of temozolomide, DLPC, DMPC, mannitol, and sodium acetate.

U.S. Pat. Nos. 6,987,108 and 7,786,118 disclose temozolomide compositions and at least one aqueous diluent. The diluent may be urea, L-histidine, L-threonine, L-asparagine, L-serine, and L-glutamine. Additional excipients include PEG.

U.S. Pat. No. 8,623,868 reports lyophilized temozolomide powder further comprising L-threonine.

WO 2021/229442 reports stable formulations of temozolomide comprising formic acid, PEG, glycerol.

Trissel, et al. (Int. J. Pharm. Compd. September-October 2006; 10(5): 396-9) describes oral temozolomide suspensions comprising ORA-Sweet® or ORA Sweet SF®, both of which comprise glycol.

U.S. Pat. No. 5,635,517 first reported lenalidomide and compositions thereof, including isotonic saline solutions. U.S. Pat. No. 7,119,106 reports pharmaceutical compositions comprising lenalidomide.

### SUMMARY

This section provides a general summary of disclosure, and is not a comprehensive disclosure of its full scope or all of its features.

The present disclosure provides compositions comprising an active pharmaceutical ingredient (API), a medium chain triglyceride (MCT), silicon dioxide, a surfactant, and optionally one or more additional excipients, wherein the API can be temozolomide or lenalidomide, wherein the composition is substantially free of water and anti-microbial preservatives, and wherein the composition is an oral suspension. In an embodiment, the compositions further comprise a flavoring agent and/or an antioxidant. The disclosed compositions are stable for shipment and storage and suitable for oral administration.

In another embodiment, the present disclosure provides methods or use of treating cancer comprising administering to a subject in need thereof a composition described herein. In an embodiment, the temozolomide or lenalidomide formulations can be administered to children, dysphagic patients, and/or patients having difficulty swallowing oral solid dosage forms.

In yet another embodiment, the present disclosure provides a method for preparing a composition comprising: adding silicon dioxide, a sweetener, a surfactant, a flavoring agent, and an API to MCT; and mixing the components until homogenous.

### BRIEF DESCRIPTION OF THE DRAWINGS

The drawings described herein are for illustrative purposes only of selected embodiments and not all possible implementations, and are not intended to limit the scope of the present disclosure.

FIG. 1 depicts the dissolution profile in 0.1 N HCl of Embodiment 1.

FIG. 2 depicts the dissolution profile in 0.1 N HCl of Embodiment 2.

FIG. 3 depicts the dissolution profile in 0.1 N HCl of Embodiment 3.

FIG. 4 depicts the XRD diffractogram of Embodiment 1 after 6 months storage at 2-8° C.

FIG. 5 depicts XRD diffractogram of Embodiment 4 after 6 months storage at 2-8° C.

FIG. 6 depicts the XRD diffractogram of Embodiment 1 after 6 months storage at 25° C./60% RH.

FIG. 7 depicts the XRD diffractogram of Embodiment 4 after 6 months storage at 25° C./60% RH.

FIG. 8 depicts the dissolution profile in 0.1 N HCl of Embodiment 5.

FIG. 9 depicts the dissolution profile in 0.1 N HCl of Embodiment 6.

FIG. 10 depicts the dissolution profile in 0.1 N HCl of Embodiment 7.

### DETAILED DESCRIPTION

The following description is merely exemplary in nature and is not intended to limit the present disclosure, application, or uses.

The term “temozolomide” refers to the chemical compound 3,4-dihydro-3-methyl-4-oxoimidazo[5,1-d]-as-tetrazine-8-carboxamide, and is also known by the trade names of Temodal®, Temodar®, and Temcad.

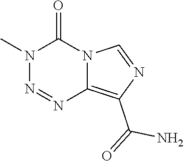

Unless otherwise noted, temozolomide includes the compound itself and pharmaceutically acceptable salts thereof.

The term “lenalidomide” refers to the chemical compound 1-oxo-2-(2,6-dioxopiperidin-3-yl)-4-aminoisoindoline, and is also known by the trade name of Revlimid®.

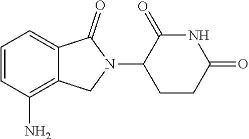 [SMILES: Nc1cccc2c1CN(C1CCC(=O)NC1=O)C2=O]

Unless otherwise noted, lenalidomide includes the compound itself and pharmaceutically acceptable salts thereof.

The term “non-aqueous” refers to compositions (e.g. liquids or suspensions) which are free of or essentially free of water. Non-aqueous is synonymous with “water-free.” The term “subject” refers to an animal that can receive administration of the temozolomide or lenalidomide composition. In some embodiment, the subject is human. In particular embodiments, the subject is a child, including newborns, infants, toddlers, adolescents, and teenagers. In another embodiment, the subject suffers from dysphagia and/or has difficulty swallowing.

The term “excipient” refers to compounds or substances that are not pharmaceutically and/or therapeutically active. Excipients are well known in the art and include, but are not limited to, carriers, vehicles, solvents, antioxidants, surfactants, suspending agents, sweeteners, flavoring agents, and combinations thereof.

The term “sweetener” refers to compositions that increase or otherwise enhance the oral sensation of sweetness. The sweetener is not particularly limited and may include sucralose, aspartame, acesulfame potassium, saccharin, saccharin sodium, neotame, and combinations thereof.

The “surfactant” is not particularly limited and may be one or more of caprylocaproyl polyoxyl-8 glycerides (LABRASOL®), including aldehyde-free caprylocaproyl polyoxyl-8 glycerides (LABRASOL®ALF), polyoxyl 15 Hydroxystearate (Kolliphor® HS-15), polyoxyl castor oil (Kolliphor® RH40), poloxamer 407 (Kolliphor® P407), poloxamer 188 (Kolliphor® P188), lauryl polyoxyl-32 glycerides (Gelucire® 44/14), polyethylene glycol sorbitan monooleate (Tween 80® or polysorbate 80) polyethylene glycol sorbitan monolaurate (Tween 20® or polysorbate 20), sorbitan monolaurate (Span®20), vitamin E polyethylene glycol succinate, and sodium dioctyl sulfosuccinate.

The term “aldehyde-free” refers to caprylocaproyl polyoxyl-8 glycerides that is free or substantially free of an aldehyde. For example, the caprylocaproyl polyoxyl-8 glycerides may be considered aldehyde-free if the caprylocaproyl polyoxyl-8 glycerides comprise no aldehyde or an aldehyde is present in an amount of less than about 95 ppm, less than about 90 ppm, less than about 85 ppm, less than about 80 ppm, less than about 75 ppm, less than about 70 ppm, less than about 65 ppm, less than about 60 ppm, less than about 55 ppm, less than about 50 ppm, less than about 45 ppm, less than about 40 ppm, less than about 35 ppm, less than about 30 ppm, less than about 25 ppm, less than about 20 ppm, less than about 15 ppm, less than about 10 ppm, less than about 5 ppm, or less than about 1 ppm. Alternatively, the caprylocaproyl polyoxyl-8 glycerides may be aldehyde-free if the percentage of aldehydes in the composition is in an amount of less than about 0.0095%, less than about 0.009% less than about 0.0085%, less than about 0.008%, less than about 0.0075%, less than about 0.007%, less than about 0.0065%, less than about 0.006%, less than about 0.0055%, less than about 0.005%, less than about 0.0045%, less than about 0.004%, less than about 0.0035%, less than about 0.003%, less than about 0.0025%, less than about 0.002%, less than about 0.0015%, less than about 0.001%, less than about 0.0005%, or less than about 0.0001%.

The term “flavoring agent” refers to compounds that render the temozolomide formulation and/or the lenalidomide formulation more palatable to oral administration. The flavoring agent is not particularly limited and may be cherry, raspberry, mint, spearmint, peppermint, orange, caramel, tutti frutti, grape, citrus, lemon, blackcurrant, tropical fruit punch, bubblegum, lime, strawberry, butterscotch, berry, cola, and a combination thereof.

The “antioxidant” is not particularly limited and may be one or more of ascorbyl palmitate, butylated hydroxyanisole (BHA), butylated hydroxytoluene (BHT), propyl gallate, and alpha-tocopherol.

As used herein, a composition is “free” of a component (e.g., free of water) when none of the component is present in the composition (e.g., 0% water). A composition is “substantially free” of a component (e.g., substantially free of water) if the component represents less than 0.5% of the total weight (e.g., <0.5% (w/w)) or less than 0.5% of the total volume (e.g., <0.5% (w/v)). In particular, a composition is “substantially free” of a component if the component represents less 0.4%, less than 0.3%, less than 0.2%, or less than 0.1% of the total weight or total volume of the composition. In a preferred embodiment, the composition disclosed herein is “substantially free” of a component if that component represents less than 0.1% of the total weight or total volume of the composition.

### Compositions

The present disclosure provides a composition comprising an API and one or more excipients, wherein the API may be temozolomide or its derivative, such as a metabolite. Alternatively, the API may be lenalidomide or its derivative, such as a metabolite. Such compositions may comprise temozolomide or lenalidomide along with MCT, silicon dioxide, and a surfactant. Temozolomide or lenalidomide is the active ingredient of the formulation and either amount can be adjusted. Generally, all known/approved amounts of temozolomide or lenalidomide can be used with the formulation.

In an embodiment, the temozolomide is present in an amount of about 1 to about 500 mg. In a further embodiment, the temozolomide is present in an amount of about 2.5 to about 250 mg. Alternatively, the temozolomide may be present at a concentration of between about 5 to about 100 mg/mL, or any interval or value therebetween, such as between about 20 and about 40 mg/mL. The temozolomide may be present at a particular amount, such as at about 1 mg/mL, at about 2.5 mg/mL, at about 5 mg/mL, at about 10 mg/mL, at about 20 mg/mL, about 30 mg/mL, about 40 mg/mL, about 50 mg/mL, about 60 mg/mL, about 70 mg/mL, about 80 mg/mL, about 90 mg/mL, or about 100 mg/mL. In a specific embodiment, the temozolomide is present in an amount of about 40 mg/mL. In yet another alternative embodiment, the temozolomide may be present in a concentration of about 0.1 to about 10% (w/v), such as between about 2 and about 4% (w/v), or at a specific concentration, such as at about 0.1% (w/v), at about 0.25% (w/v), at about 0.5% (w/v), at about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). The temozolomide may be micronized or unmicronized.

In an embodiment, the lenalidomide is present in an amount of about 0.1 to about 100 mg. In a further embodiment, the lenalidomide is present in an amount of about 0.25 to about 50 mg. Alternatively, the lenalidomide may be present at a concentration of between about 0.1 to about 100 mg/mL, or any interval or value therebetween, such as between about 0.25 and about 50 mg/mL. The lenalidomide may be present at a particular amount, such as at about 0.1 mg/mL, about 0.25 mg/mL, about 0.5 mg/mL, about 1 mg/mL, about 2.5 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL. In a specific embodiment, the lenalidomide is present in an amount of about 5 mg/mL. In yet another alternative embodiment, the lenalidomide may be present in a concentration of about 0.01 to about 10% (w/v), such as between about 0.025 and about 5% (w/v), or at a specific concentration, such as at about 0.01% (w/v), about 0.025% (w/v), about 0.05% (w/v), about 0.1% (w/v), about 0.25% (w/v), about 0.5% (w/v), about 1% (w/v), about 2.5% (w/v), about 5% (w/v), or about 10% (w/v). The lenalidomide may be micronized or unmicronized.

In an embodiment, the MCT may be present in an amount of about 400 to about 1,200 mg/mL, alternatively, about 400 to about 980 mg/mL, and any value or interval therebetween. In another embodiment, the MCT may be present in an amount of about 850 to about 950 mg/mL. In a particular embodiment, the MCT may be present in an amount of about 850 mg/mL, about 860 mg/mL, about 870 mg/mL, about 880 mg/mL, about 890 mg/mL, about 900 mg/mL, about 910 mg/mL, about 920 mg/mL, about 930 mg/mL, about 940 mg/mL, about 950 mg/mL, about 960 mg/mL, about 970 mg/mL, about 975 mg/mL, or about 980 mg/mL, and all values therebetween. In a particular embodiment, the MCT may be MIGLYOL®. In some embodiments, the MIGLYOL® may be MIGLYOL® 812N or MIGLYOL® 810.

In an embodiment, the silicon dioxide may be present in an amount of between about 1 to about 20 mg/mL and any value or interval therebetween, such as between about 10 and about 15 mg/mL. In a particular embodiment, the silicon dioxide may be present in an amount of about 12.1 mg/mL to about 12.5 mg/ml.

In an embodiment, the surfactant may be present in an amount of between 20 and about 70 mg/mL and any value or interval therebetween, such as between about 40 mg/mL and about 50 mg/mL. In a particular embodiment, the surfactant may be caprylocaproyl poloxyl-8 glycerides (e.g., LABRASOL® ALF), polysorbate 80 (Tween 80), polysorbate 20 (Tween 20), sorbitan monolaurate (Span 20), or vitamin E polyethylene glycol succinate (PGS).

In a further embodiment, the composition may further comprise a sweetener and/or a flavoring agent. The sweetener may be present in an amount of between about 0.1 to about 3 mg/mL, and any value or interval therebetween. In a particular embodiment, the sweetener may be present in an amount of about 1.9 mg/mL to 2.0 mg/ml. The sweetener may be sucralose, aspartame, acesulfame potassium, saccharin, saccharin sodium, or neotame. In particular, the sweetener may be sucralose.

The flavoring agent may be present in an amount of between about 0.1 to about 5 mg/mL, and any interval or value therebetween. In particular, the flavoring agent may be present in an amount of about 2.9 mg/mL to about 3.0 mg/ml. The flavoring agent may be grape flavoring, cola flavoring, cherry flavoring, berry flavoring, raspberry flavoring, mint flavoring, orange flavoring, caramel flavoring, and tutti frutti. In a specific embodiment, the flavoring agent may be grape flavoring.

In another embodiment, the composition may further comprise an antioxidant. When present, the antioxidant may be present in an amount of between about 0.05 to about 2 mg/mL. and any value or interval therebetween. In a particular embodiment, the antioxidant may be present in an amount of about 0.2 to about 0.5 mg/mL. The antioxidant may be butylated hydroxytoluene (BHT), butylated hydroxyanisole (BHA), ascorbyl palmitate, or propyl gallate.

The composition described herein may be in the form of a suspension. It may be formulated as a pharmaceutical composition for administration to a subject. The composition may be in a single-dose formulation or in a multidose formulation. The formulation may be non-aqueous, wherein the formulation is water-free (e.g., devoid of water) or essentially water-free (e.g., effectively devoid of water). The composition may be formulated in the form of a non-aqueous suspension for oral administration. Additionally and alternatively, the composition may be free or substantially free of a preservative (e.g., preservative free). The composition may also be free or substantially free of an organic solvent (e.g., ethanol or propylene glycol) and/or an acid.

In general, the composition described herein may comprise about 5 to about 100 mg/mL temozolomide; about 400 to about 975 mg/mL MCT (e.g., MIGLYOL®); about 1 to about 20 mg/mL silicon dioxide; about 0.1 to about 3 mg/mL sweetener; about 20 to about 70 mg/mL surfactant; and about 0.1 to about 5 mg/mL flavoring agent. In another embodiment, the composition may comprise about 5 to about 100 mg/mL temozolomide; about 400 to about 960 mg/mL MCT (e.g., MIGLYOL®); about 1 to about 20 mg/mL silicon dioxide; about 0.1 to about 3 mg/mL sweetener; about 20 to about 70 mg/mL surfactant; about 0.1 to about 5 mg/mL flavoring agent; and about 0.05 to about 2 mg/mL antioxidant. Additionally or alternatively, the composition described herein may consist essentially of about 5 to about 100 mg/mL temozolomide; about 400 to about 975 mg/mL MCT (e.g., MIGLYOL®); about 1 to about 20 mg/mL silicon dioxide; about 0.1 to about 3 mg/mL sweetener; about 20 to about 70 mg/mL surfactant; and about 0.1 to about 5 mg/mL flavoring agent. In another embodiment, the composition may consist essentially of about 5 to about 100 mg/mL temozolomide; about 400 to about 975 mg/mL MCT (e.g., MIGLYOL®); about 1 to about 20 mg/mL silicon dioxide; about 0.1 to about 3 mg/mL sweetener; about 20 to about 70 mg/mL surfactant; about 0.1 to about 5 mg/mL flavoring agent; and about 0.05 to about 2 mg/mL antioxidant.

In general, the composition described herein may comprise about 0.25 to about 50 mg/mL lenalidomide; about 450 to about 980 mg/mL MCT (e.g., MIGLYOL®); about 1 to about 20 mg/mL silicon dioxide; about 0.1 to about 3 mg/mL sweetener; about 20 to about 70 mg/mL surfactant; and about 0.1 to about 5 mg/mL flavoring agent. In another embodiment, the composition may comprise about 0.25 to about 50 mg/mL lenalidomide; about 450 to about 980 mg/mL MCT (e.g., MIGLYOL®); about 1 to about 20 mg/mL silicon dioxide; about 0.1 to about 3 mg/mL sweetener; about 20 to about 70 mg/mL surfactant; about 0.1 to about 5 mg/mL flavoring agent; and about 0.05 to about 2 mg/mL antioxidant. Additionally or alternatively, the composition described herein may consist essentially of about 0.25 to about 50 mg/mL lenalidomide; about 450 to about 980 mg/mL MCT (e.g., MIGLYOL®); about 1 to about 20 mg/mL silicon dioxide; about 0.1 to about 3 mg/mL sweetener; about 20 to about 70 mg/mL surfactant; and about 0.1 to about 5 mg/mL flavoring agent. In another embodiment, the composition may consist essentially of about 0.25 to about 50 mg/mL lenalidomide; about 450 to about 980 mg/mL MCT (e.g., MIGLYOL®); about 1 to about 20 mg/mL silicon dioxide; about 0.1 to about 3 mg/mL sweetener; about 20 to about 70 mg/mL surfactant; about 0.1 to about 5 mg/mL flavoring agent; and about 0.05 to about 2 mg/mL antioxidant.

Specific exemplary formulations are shown in Table 1 below. In one particular embodiment, the composition comprises about 40 mg/mL temozolomide; about 868.7 mg/mL MCT; about 12.1 mg/mL silicon dioxide; about 1.9 mg/mL sucralose; about 44.4 mg/mL caprylocaproyl poloxyl-8 glycerides; and about 2.9 mg/mL grape flavoring agent (Embodiment 1). In another particular embodiment, the composition comprises about 40 mg/mL temozolomide; about 868.5 mg/mL MCT; about 12.1 mg/mL silicon dioxide; about 1.9 mg/mL sucralose; about 44.4 mg/mL caprylocaproyl poloxyl-8 glycerides; about 2.9 mg/mL grape flavoring agent; and about 0.2 mg/mL BHT (Embodiment 2). In another particular embodiment, the composition comprises about 40 mg/mL temozolomide; about 868.4 mg/mL MCT; about 12.1 mg/mL silicon dioxide; about 1.9 mg/mL sucralose; about 44.4 mg/mL caprylocaproyl poloxyl-8 glycerides; about 2.9 mg/mL grape flavoring agent; and about 0.3 mg/mL BHT (Embodiment 3). In another particular embodiment, the composition comprises about 40 mg/mL temozolomide; about 880.8 mg/mL MCT; about 1.9 mg/mL sucralose; about 44.4 mg/mL caprylocaproyl poloxyl-8 glycerides; and about 2.9 mg/mL grape flavoring agent (Embodiment 4). Embodiment 4 lacks silicon dioxide and is used as a comparative formulation.

Specific exemplary formulations are shown in Table 15 below. In one particular embodiment, the composition comprises about 5.0 mg/mL lenalidomide; about 902.5 mg/mL MCT; about 12.5 mg/mL silicon dioxide; about 2.0 mg/mL sucralose; about 45.0 mg/mL caprylocaproyl poloxyl-8 glycerides; and about 3.0 mg/mL grape flavoring agent (Embodiment 5). In another particular embodiment, the composition comprises about 5.0 mg/mL lenalidomide; about 902.0 mg/mL MCT; about 12.5 mg/mL silicon dioxide; about 2.0 mg/mL sucralose; about 45.0 mg/mL caprylocaproyl poloxyl-8 glycerides; about 0.5 mg/mL BHT, and about 3.0 mg/mL grape flavoring agent (Embodiment 6). In another particular embodiment, the composition comprises about 5.0 mg/mL lenalidomide; about 902.5 mg/mL MCT; about 12.5 mg/mL silicon dioxide; about 2.0 mg/mL sucralose; about 45.0 mg/mL caprylocaproyl poloxyl-8 glycerides; and about 3.0 mg/mL grape flavoring agent (Embodiment 7).

In another embodiment, the temozolomide or lenalidomide compositions are in the form of a suspension. In a further embodiment, the temozolomide or lenalidomide compositions are a suspension which is substantially free of water and/or substantially free of anti-microbial preservatives. In a further embodiment, the composition may be substantially free of an organic solvent (e.g., ethanol and propylene glycol) and/or acid.

The composition of the present disclosure is to address, among other things, stability issues of temozolomide or lenalidomide in an aqueous solution. The stable temozolomide composition of the present disclosure is suitable for a ready-to-use liquid dosage drug product. In an embodiment, the temozolomide composition is stable at a refrigeration temperature such as e.g., about 4-5° C. for one month, two months, three months, or longer. In another embodiment, the temozolomide is stable for at least six months under refrigeration. In an alternative embodiment, the temozolomide composition is stable at room temperature (e.g., about 21-25° C.) for one month, two months, three months, four months, six months or longer. In another embodiment, the lenalidomide composition is stable at a refrigeration temperature for one month, two months, three months, or longer. In another embodiment, the lenalidomide is stable for at least six months at a refrigeration temperature. In an alternative embodiment, the lenalidomide composition is stable at room temperature (e.g., about 21-25° C.) for one month, two months, three months, four months, six months or longer.

Additionally or alternatively, the temozolomide or lenalidomide compositions are stable at elevated temperature (e.g., 40° C.) for one week, two weeks, three weeks, four weeks, one month, or longer. In some embodiments, the temozolomide composition may show less than 0.8% Impurity E, less than 1.0% Impurity A, less than 0.2% single largest unknown impurity (SLU) and less than 1.2% total impurities following storage under refrigerated conditions (e.g., 4-5° C.), may show less than 0.8% Impurity E, less than 1.0% Impurity A, less than 0.2% single largest unknown impurity and less than 1.2% total impurities following storage at room temperature (e.g., 25° C.), or less than 0.8% Impurity E, less than 1.0% Impurity A, less than 0.2% single largest unknown impurity and less than 1.2% total impurities following storage at 40° C. Additionally or alternatively, the temozolomide may be in a particular polymorphic form when initially prepared in suspension. Following storage for a period of time (e.g., for up to six months or longer), the temozolomide may remain in the same polymorphic form as it was at the initial storage time.

In other embodiments, the lenalidomide composition shows less than 0.2% of the single largest unknown impurity (SLU) and less than 1.0% total impurities under refrigerated conditions (e.g., 4-5° C.), shows less than 0.2% SLU and less than 1.0% total impurities following storage at room temperature (e.g., 25° C.), or less than 0.2% SLU and less than 1.0% total impurities following storage at 40° C. Additionally or alternatively, the lenalidomide can be in a particular polymorphic form when initially prepared in suspension. Following storage for a period of time (e.g., for up to six months or longer), the lenalidomide may remain in the same polymorphic form as it was at the initial storage time.

### Methods of Treatment

The present disclosure provides various methods of using the temozolomide or lenalidomide compositions for the treatment of disease(s) such as cancer. In an embodiment, the temozolomide composition is administered to a subject to treat cancer, wherein the subject is in need of such treatment. Various cancers can be treated by the temozolomide composition and in some embodiments, the cancer is glioblastoma multiforme (GBM), including newly diagnosed GBM, or anaplastic astrocytoma, including refractory anaplastic astrocytoma. In a specific embodiment, the GBM may be newly diagnosed GBM and the anaplastic astrocytoma may be refractory anaplastic astrocytoma.

In an embodiment, the lenalidomide composition is administered to a subject to treat cancer, wherein the subject is in need of such treatment. Various cancers can be treated by the lenalidomide composition and in some embodiments, the cancer is multiple myeloma, myelodysplastic syndromes, mantle cell lymphoma, follicular lymphoma, and marginal zone lymphoma.

The temozolomide or lenalidomide composition therapies can be combined with various cancer treatments known in the art. In an embodiment, the temozolomide composition is administered to the subject in conjunction with radiotherapy. In still another embodiment, the temozolomide composition or lenalidomide composition is administered before, after, or concurrently with an additional chemotherapeutic agent. Non-limiting examples of such additional chemotherapeutic agents include nitrosourea and procarbazine.

The temozolomide composition or lenalidomide composition therapy is contemplated for pediatric use. In a particular embodiment, the subject is a child.

The temozolomide composition or lenalidomide composition therapy is also contemplated for subjects suffering from dysphagia and for non-dysphagic patients.

### EXAMPLES

### Example 1: Temozolomide Compositions

Temozolomide formulations were prepared as follows: The temozolomide suspensions were prepared under a nitrogen atmosphere. For Embodiments 1 and 4 (which lack an antioxidant), MIGLYOL® 812N was weighed to the appropriate weight. Silicon dioxide (as Aerosil 200P™) (in the case of embodiment 1), sucralose, grape flavor, LABRASOL® ALF and temozolomide were added at the appropriate weights and mixed until homogenous. For Embodiments 2 and 3 (which include an antioxidant), MIGLYOL® 812N was weighed to the appropriate weight. Silicon dioxide (as Aerosil 200P™), sucralose, and grape flavor were mixed into the MIGLYOL®. LABRASOL® ALF and BHT were mixed until BHT was fully dissolved. Temozolomide and the LABRASOL® ALF-BHT mixture were then added to the MIGLYOL® suspension and mixed until homogenous. The concentrations of each component are listed in Table 1 below. The temozolomide formulations were dispensed into 30 mL Alpha amber glass bottles at a volume of 27.5 mL. The bottles were secured with a child-resistant, tamper-evident PP28 cap, complete with a low density polyethylene syringe adaptor.

TABLE 1

| Material | Em. 1 | | Em. 2 | | Em. 3 | | Em. 4 | |
|---|---|---|---|---|---|---|---|---|
| TMZ | 40.0 | mg/mL | 40.0 | mg/ml | 40.0 | mg/mL | 40.0 | mg/mL |
| Miglyol ® 812 | 868.7 | mg/mL | 868.5 | mg/mL | 868.4 | mg/mL | 880.8 | mg/mL |
| Silicon Dioxide | 12.1 | mg/mL | 12.1 | mg/mL | 12.1 | mg/mL | 0.00 | mg/mL |
| Sucralose | 1.9 | mg/mL | 1.9 | mg/mL | 1.9 | mg/mL | 1.9 | mg/mL |
| Labrasol ® ALF | 44.4 | mg/mL | 44.4 | mg/mL | 44.4 | mg/mL | 44.4 | mg/mL |
| Grape flavor | 2.9 | mg/mL | 2.9 | mg/mL | 2.9 | mg/mL | 2.9 | mg/mL |
| BHT | 0.00 | mg/mL | 0.2 | mg/mL | 0.3 | mg/mL | 0.00 | mg/mL |

### Example 2: Stability of Temozolomide Formulations

Embodiments 1-4 were prepared as described in Example 1 and stored at either 2-8° C., 25° C./60% RH, or 40° C./75% RH. The compositions were tested for impurities at regular intervals (e.g., after one, two, three, and six months). Impurities include known temozolomide degradation products, such as Impurity A (5-amino-1H-imidazole-4-carboxamide; generated via acidic, alkaline, or thermal hydrolysis of temozolomide) and Impurity E (3,7-dihydro-4H-imidazo[4,5-d][1,2,3]triazin-4-one; generated via oxidation of temozolomide). The compositions described in Example 1 were tested for impurities at 1, 2, 3 and 6 months. Tables 2-7 below show the relative stability of Embodiments 1-4. Embodiment 4 lacks silicon dioxide and is used as a comparative formulation. (For all tables, ND=not detected and LOQ means limit of quantitation.)

TABLE 2
Stability at 25° C./60% RH (initial and month 1)-presence of
impurities

| | Initial | | | | Month 1 (25° C./60% RH) | | | |
|---|---|---|---|---|---|---|---|---|
| Em. | Imp A | Imp E | SLU* | Tot** | Imp A | Imp E | SLU* | Tot** |
| 1 | ND | ND | ND | 0.0 | ND | 0.10 | ND | 0.10 |
| 2 | ND | ND | ND | 0.0 | ND | 0.07 | ND | 0.07 |
| 3 | ND | ND | ND | 0.0 | ND | 0.09 | ND | 0.09 |
| 4 | ND | 0.05 | ND | 0.05 | 0.06 | 0.33 | ND | 0.40 |

*Single largest unknown impurity
**Total impurities

TABLE 3
Stability at 25° C./60% RH (month 2 and month 3)-presence of
impurities

| | Month 2 (25° C./60% RH) | | | | Month 3 (25° C./60% RH) | | | |
|---|---|---|---|---|---|---|---|---|
| Em. | Imp A | Imp E | SLU* | Tot** | Imp A | Imp E | SLU* | Tot** |
| 1 | ND | 0.09 | ND | 0.09 | <LOQ | 0.26 | ND | 0.26 |
| 2 | ND | 0.08 | ND | 0.08 | <LOQ | 0.21 | ND | 0.21 |
| 3 | ND | 0.11 | ND | 0.11 | 0.05 | 0.28 | ND | 0.33 |

*Single largest unknown impurity
**Total impurities

TABLE 4
Stability at 25° C./60% RH (initial and month 6)-presence of
impurities

| | Initial | | | | Month 6 (25° C./60% RH) | | | |
|---|---|---|---|---|---|---|---|---|
| Em. | Imp A | Imp E | SLU* | Tot** | Imp A | Imp E | SLU* | Tot** |
| 1 | ND | ND | ND | 0.0 | 0.21 | 0.59 | 0.10 | 0.97 |
| 2 | ND | ND | ND | 0.0 | 0.15 | 0.50 | 0.10 | 0.75 |
| 3 | ND | ND | ND | 0.0 | 0.28 | 0.68 | 0.12 | 1.08 |
| 4 | ND | 0.05 | ND | 0.05 | 0.87 | 1.06 | 0.49 | 2.75 |

*Single largest unknown impurity
**Total impurities

TABLE 5
Stability at 40° C./75 % RH (initial and month 1)-presence of
impurities

| | Initial | | | | Month 1 (40° C./75% RH) | | | |
|---|---|---|---|---|---|---|---|---|
| Em. | Imp A | Imp E | SLU* | Tot** | Imp A | Imp E | SLU* | Tot** |
| 1 | ND | ND | ND | 0.0 | 0.27 | 0.71 | 0.07 | 1.05 |
| 2 | ND | ND | ND | 0.0 | 0.23 | 0.51 | ND | 0.74 |
| 3 | ND | ND | ND | 0.0 | 0.31 | 0.66 | ND | 0.97 |
| 4 | ND | 0.05 | ND | 0.05 | 0.67 | 0.96 | ND | 1.63 |

*Single largest unknown impurity
**Total impurities

TABLE 6
Stability at 2-8° C. (initial and month 3)-presence of impurities

| | Initial | | | | Month 3 (2-8° C.) | | | |
|---|---|---|---|---|---|---|---|---|
| Em. | Imp A | Imp E | SLU* | Tot** | Imp A | Imp E | SLU* | Tot** |
| 1 | ND | ND | ND | 0.0 | ND | <LOQ | ND | 0.0 |
| 2 | ND | ND | ND | 0.0 | ND | <LOQ | ND | 0.0 |
| 3 | ND | ND | ND | 0.0 | ND | <LOQ | ND | 0.0 |

*Single largest unknown impurity
**Total impurities

TABLE 7
Stability at 2-8° C. (initial and month 6)-presence of impurities

| | Initial | | | | Month 6 (2-8° C.) | | | |
|---|---|---|---|---|---|---|---|---|
| Em. | Imp A | Imp E | SLU* | Tot** | Imp A | Imp E | SLU* | Tot** |
| 1 | ND | ND | ND | 0.0 | ND | <LOQ | ND | 0.0 |
| 2 | ND | ND | ND | 0.0 | ND | ND | ND | 0.0 |
| 3 | ND | ND | ND | 0.0 | ND | ND | ND | 0.0 |

*Single largest unknown impurity
**Total impurities

The data shown above illustrate that the temozolomide formulations described herein are stable under various conditions. The stability is due, in part, to the presence of the silicon dioxide and the absence of water. The enhanced stability is seen in the absence of any added preservative, indicating that a preservative is not necessary for stable storage.

### Example 3: In-Use Stability of Freshly Manufactured Samples of Temozolomide Oral Suspension

An in-use stability study was performed on freshly manufactured samples of Embodiments 1-3. The study involved taking a bottle of each Embodiment, removing and analyzing a sample on the day of first opening (TO), after seven days, after fourteen days and after thirty days. The sampling procedure for each sample involved vigorously shaking the sample bottle for 20 seconds, then immediately removing the child-resistant cap and extracting the necessary volume for analysis with a syringe through the adaptor. On days not designated for analysis (days 1-6 after opening, days 8-13 after opening and days 15-29 after opening), patient handling was simulated as follows: the bottle was shaken vigorously for 20 seconds, the child-resistant cap was immediately removed and sample withdrawal by a syringe through the adaptor was mimicked. The bottle was stored at 2-8° C. for the 30 day duration of the in-use study.

TABLE 8
In-use stability of freshly manufactured sample
of Embodiment 1 at 2-8° C.-presence of impurities

Time point

| | | | |
|---|---|---|---|
| T0 | 7 d | 14 d | 30 d |

Result %

| | | | | |
|---|---|---|---|---|
| Imp A | <0.05 | <0.05 | <0.05 | <0.05 |
| Imp E | <0.05 | <0.05 | <0.05 | <0.05 |
| SLU* | <0.05 | <0.05 | <0.05 | 0.16 |
| Total impurities | <0.05 | <0.05 | <0.05 | 0.16 |

*Single largest unknown impurity

TABLE 9
In-use stability of freshly manufactured sample
of Embodiment 2 at 2-8° C.-presence of impurities

Timepoint

| | | | |
|---|---|---|---|
| T0 | 7 d | 14 d | 30 d |

Result %

| | | | | |
|---|---|---|---|---|
| Imp A | <0.05 | <0.05 | <0.05 | <0.05 |
| Imp E | <0.05 | <0.05 | <0.05 | <0.05 |
| SLU* | <0.05 | <0.05 | <0.05 | 0.13 |
| Total impurities | <0.05 | <0.05 | <0.05 | 0.13 |

*Single largest unknown impurity

TABLE 10
In-use stability of freshly manufactured sample
of Embodiment 3 at 2-8° C.-presence of impurities

Timepoint

| | | | |
|---|---|---|---|
| T0 | 7 d | 14 d | 30 d |

Result %

| | | | | |
|---|---|---|---|---|
| Imp A | <0.05 | <0.05 | <0.05 | <0.05 |
| Imp E | <0.05 | <0.05 | <0.05 | <0.05 |
| SLU* | <0.05 | <0.05 | <0.05 | 0.07 |
| Total impurities | <0.05 | <0.05 | <0.05 | 0.07 |

*Single largest unknown impurity

The data shown above in Tables 8-10 illustrate that the temozolomide formulations described herein are stable across the in-use period of 30 days when stored at 2-8° C.

### Example 4: In-Use Stability on Samples Stored for Six Months—Temozolomide Oral Suspension

An in-use stability study was conducted on Embodiment 1, after storage for six months at 2-8° C. In addition, an in-use stability study was conducted on Embodiment 1, after storage for six months at 25° C./60% RH. These studies involved selecting bottles of Embodiment 1 which had been previously stored as outlined above, removing, and analyzing samples on the first day of opening (TO), after seven days, after fourteen days and after thirty days.

The sampling procedure involved vigorously shaking the bottle for 20 seconds, then immediately removing the child-resistant cap and extracting the necessary volume for analysis with a syringe through the adaptor. On days not designated for analysis (days 1-6 after opening, days 8-13 after opening and days 15-29 after opening), patient handling was simulated as follows: the bottle was shaken vigorously for 20 seconds, the child-resistant cap was immediately removed and sample withdrawal by a syringe through the adaptor was mimicked.

The bottle was either stored at 2-8° C. for the 30-day duration of the in-use study (if previously stored at 2-8° C. for 6 months) or at 25° C./60% RH for the 30-day duration of the in-use study (if previously stored at 25° C./60% RH for 6 months).

TABLE 11
In-use stability on Embodiment 1 at 2-8° C. after six
months storage at 2-8° C.-presence of impurities

Timepoint

| | | | |
|---|---|---|---|
| T0 | 7 d | 14 d | 30 d |

Result %

| | | | | |
|---|---|---|---|---|
| Imp A | <0.05 | <0.05 | <0.05 | <0.05 |
| Imp E | <0.05 | <0.05 | <0.05 | <0.05 |
| SLU* | <0.05 | <0.05 | <0.05 | 0.15 |
| Total impurities | <0.05 | <0.05 | <0.05 | 0.15 |

*Single largest unknown impurity

TABLE 12
In-use stability on Embodiment 1 at 25° C./60% RH
after six months storage at 25° C./60% RH

Timepoint

| | | | |
|---|---|---|---|
| T0 | 7 d | 14 d | 30 d |

Result %

| | | | | |
|---|---|---|---|---|
| Imp A | 0.1 | 0.11 | 0.12 | 0.16 |
| Imp E | 0.37 | 0.41 | 0.46 | 0.57 |
| SLU* | <0.05 | <0.05 | 0.07 | 0.07 |
| Total impurities | <0.05 | <0.05 | 0.72 | 0.86 |

*Single largest unknown impurity

The data shown above in Tables 11 and 12 illustrate that the temozolomide formulations described herein are stable across the in-use period of 30 days when stored at 2-8° C. or at 25° C./60% RH after six months storage at 2-8° C. or at 25° C./60% RH.

### Example 5: X-Ray Diffraction Analysis—Temozolomide Oral Suspension

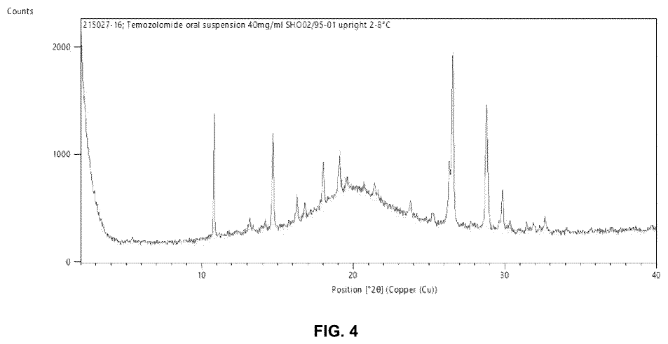

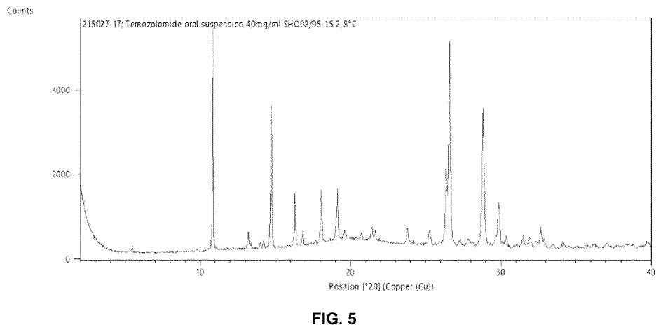

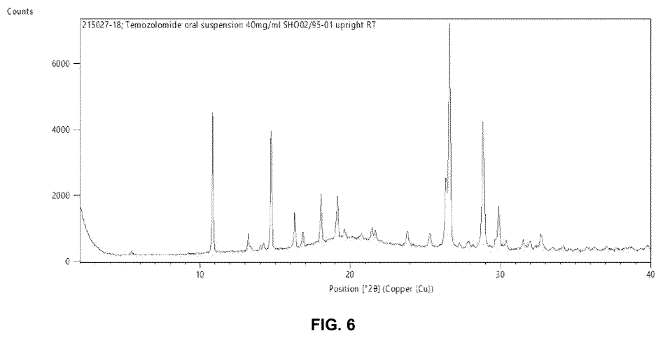

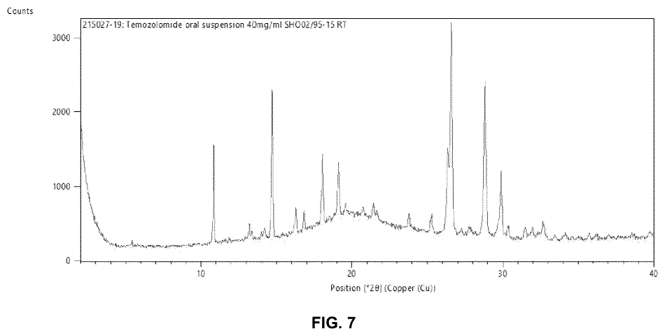

Temozolomide API Polymorph Form III was used in the manufacture of Embodiment 1 and Embodiment 4. XRD testing was conducted on Embodiment 1 and Embodiment 4 which had been stored at temperatures of 2-8° C. and 25° C./60% RH for six months. Information on the instrumentation and testing methods used is outlined in Table 13. The results of XRD testing are presented in FIGS. 4-7. The polymorphic form of temozolomide in Embodiment 1 and Embodiment 4 remained as Polymorph Form III after 6 months of storage at 2-8° C. The polymorphic form of temozolomide in Embodiment 1 and Embodiment 4 remained as Polymorph Form III after 6 months of storage at 25° C./60% RH.

TABLE 13
Instrumentation and Methods Used for X-Ray Diffraction Analysis

| Technique | Instrument | Test Method |
|---|---|---|
| Polymorph Determination by | Bruker D8 Advance XRD with LynxEye ™ | Range: 2-40 °2theta, Rotation: ON, Step Size: |
| X-Ray Diffraction (XRD) | Detector using 9 mm cavity PMMA sample holders | 0.02 °2theta, Dwell Time: 0.2 seconds, Slits: V9 |
| Sample Preparation by | Heraeus Megafuge Centrifuge | 4,500 rpm for 10 Minutes |
| Centrifugation | | |

### Example 6: Dissolution Profile

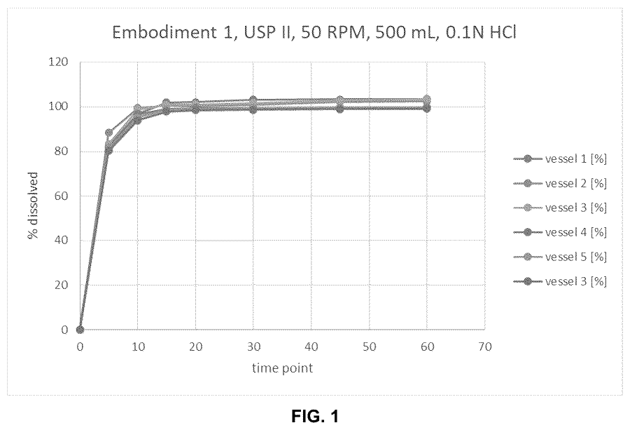

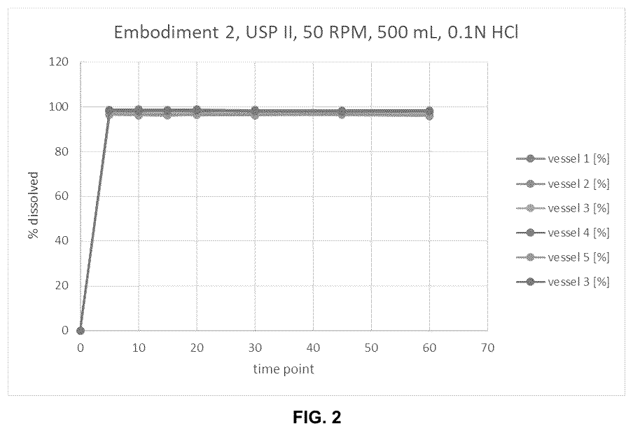

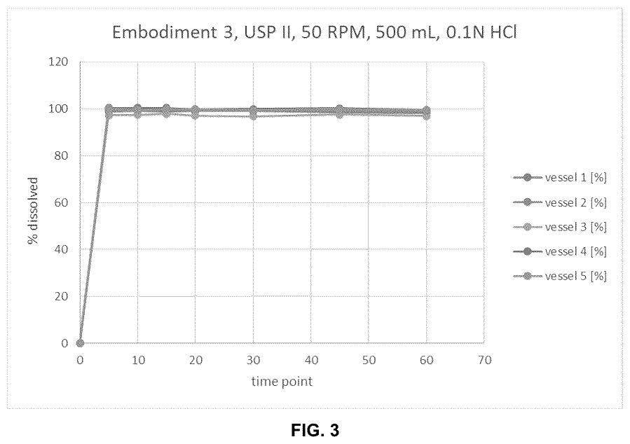

Embodiments 1-3 were prepared as described in Example 1. The dissolution profile for the embodiments was tested using USP II (Paddle) Dissolution Apparatus by placing a sample of the composition in 500 mL of 0.1 N HCl and using a paddle stir rate of 50 rpm. FIGS. 1-3 show the dissolution profile of Embodiments 1-3, respectively. Each sample of the embodiments had a consistent dissolution rate of greater than 85% dissolution within 15 minutes. This data show that the compositions described herein are rapidly dissolving.

### Example 7: Water Activity

The water activity of the temozolomide formulations relates to the effective concentration of pure water in a drug product. When applied to a non-sterile drug product, water activity is a critical physical attribute that determines whether the product will support the growth of microorganisms. The most osmophilic yeast and xerophilic fungi will not proliferate below water activity of 0.60. The temozolomide formulations described herein have a water activity result <0.60, as shown in Table 14, which does not create a suitable environment even for the most resistant microorganisms. Embodiments 1-3 were tested for water activity following storage for 3 months at 25° C./60% RH. Briefly, the water activity was measured by allowing the sample to equilibrate with the moisture in the headspace above the sample and then measuring the equilibrium relative humidity (ERH) of the headspace. At equilibrium, the ERH is also a measure of the water activity in the formulation. Water activity is measured with HygroPalm™ 23-AW, a portable water activity analyzer equipped with a probe and a sample cup with a cover for the acclimatization of the sample before measuring. The product is filled into the cup to the fill line and covered with the lid with the probe built in. The measurement is taken after approximately 5-6 minutes.

TABLE 14
Water activity

| Embodiment | Water Activity |
|---|---|
| 1 | 0.232 |
| 2 | 0.207 |
| 3 | 0.223 |

### Example 8: Lenalidomide Compositions

Lenalidomide formulations were prepared as follows: For Embodiments 5 and 7 (which exclude an antioxidant), MIGLYOL® 812N was weighed to the appropriate weight. Silicon dioxide (Aerosil 200P™), sucralose, grape flavor, LABRASOL® ALF, and Lenalidomide were added at the appropriate weights and mixed until homogeneous. The manufacturing and filling was conducted under a nitrogen atmosphere for Embodiment 5. In contrast, Embodiment 7 was manufactured and filled under atmospheric conditions.

For Embodiment 6, which includes an antioxidant, MIGLYOL® 812N was weighed to the appropriate weight. Silicon dioxide (Aerosil 200P™), sucralose, and grape flavor were added at the appropriate weights into the MIGLYOL® and mixed until homogeneous. Subsequently, LABRASOL®ALF and BHT were combined and stirred until the BHT was completely dissolved. Lenalidomide and the LABRASOL®ALF-BHT mixture were then added to the MIGLYOL® mixture and mixed until homogeneous. The manufacturing and filling processes for Embodiment 6 were conducted under a nitrogen atmosphere.

The concentrations of each component used in the formulations are detailed in Table 15. After manufacturing, the lenalidomide formulations were dispensed into 30 mL Alpha amber glass bottles, each filled to a volume of 17 mL. The bottles were secured with child-resistant, tamper-evident PP28 caps, complete with a low-density polyethylene syringe adaptor.

TABLE 15

| Material | Em. 5 | | Em. 6 | | Em. 7 | |
|---|---|---|---|---|---|---|
| Lenalidomide | 5.0 | mg/mL | 5.0 | mg/mL | 5.0 | mg/mL |
| Miglyol ® 812 | 902.5 | mg/mL | 902.0 | mg/mL | 902.5 | mg/mL |
| Silicon Dioxide | 12.5 | mg/mL | 12.5 | mg/mL | 12.5 | mg/mL |
| Labrasol ® ALF | 45.0 | mg/mL | 45.0 | mg/mL | 45.0 | mg/mL |
| BHT | 0.00 | mg/mL | 0.5 | mg/mL | 0.00 | mg/mL |
| Sucralose | 2.0 | mg/mL | 2.0 | mg/mL | 2.0 | mg/mL |
| Grape flavor | 3.0 | mg/mL | 3.0 | mg/mL | 3.0 | mg/mL |

### Example 9: Stability of Lenalidomide Formulations

Embodiments 5, 6 and 7 were prepared as in Example 8 and stored at either 2-8° C., 25° C./60% RH, or 40° C./75% RH. The compositions were tested for impurities at one month. The specifications for impurity SLU (Single Largest Unknown) and total impurities are set not to exceed 0.2% and 1.0%, respectively. These limits apply to all storage conditions including under refrigerated conditions (e.g., 4-5° C.), at room temperature (e.g., approximately 25° C.) and at elevated temperatures (e.g., around 40° C.).

TABLE 16
Stability at 2-8° C. (initial and month 1)-presence of impurities

| | |
|---|---|
| Initial | Month 1 (2-8° C.) |

| Em. | SLU* | Tot** | SLU* | Tot** |
|---|---|---|---|---|
| 5 | 0.1 | 0.1 | 0.07 | 0.07 |
| 6 | 0.1 | 0.1 | 0.07 | 0.07 |
| 7 | 0.1 | 0.1 | 0.08 | 0.08 |

*Single largest unknown impurity
**Total impurities

TABLE 17
Stability at 25° C./60% RH (initial
and month 1)-presence of impurities

| | |
|---|---|
| Initial | Month 1 (25° C./60% RH) |

| Em. | SLU* | Tot** | SLU* | Tot** |
|---|---|---|---|---|
| 5 | 0.1 | 0.1 | 0.08 | 0.08 |
| 6 | 0.1 | 0.1 | 0.08 | 0.08 |
| 7 | 0.1 | 0.1 | 0.07 | 0.07 |

*Single largest unknown impurity
**Total impurities

TABLE 18
Stability at 40° C./75% RH (initial
and month 1)-presence of impurities

| | |
|---|---|
| Initial | Month 1 (40° C./75% RH) |

| Em. | SLU* | Tot** | SLU* | Tot** |
|---|---|---|---|---|
| 5 | 0.1 | 0.1 | 0.07 | 0.07 |
| 6 | 0.1 | 0.1 | 0.08 | 0.08 |
| 7 | 0.1 | 0.1 | 0.08 | 0.08 |

*Single largest unknown impurity
**Total impurities

The data shown above illustrate that the lenalidomide formulations described herein are stable under various conditions. The stability was due, in part, to the presence of the silicon dioxide and the absence of water. The enhanced stability was seen in the absence of any added preservative, indicating that a preservative is not necessary for stable storage.

### Example 10: Dissolution Profile

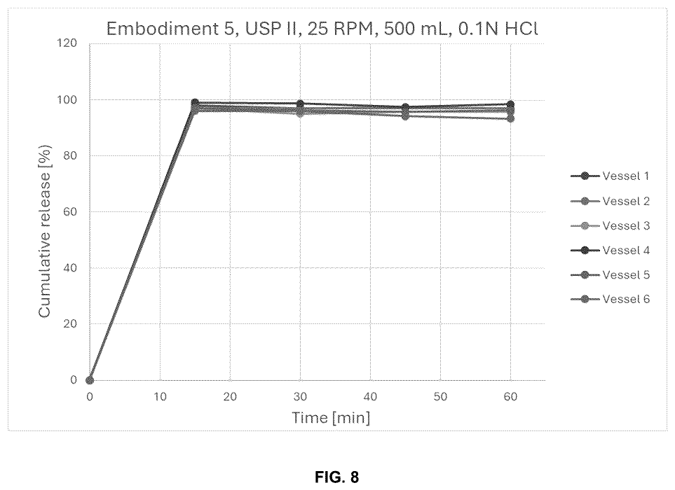

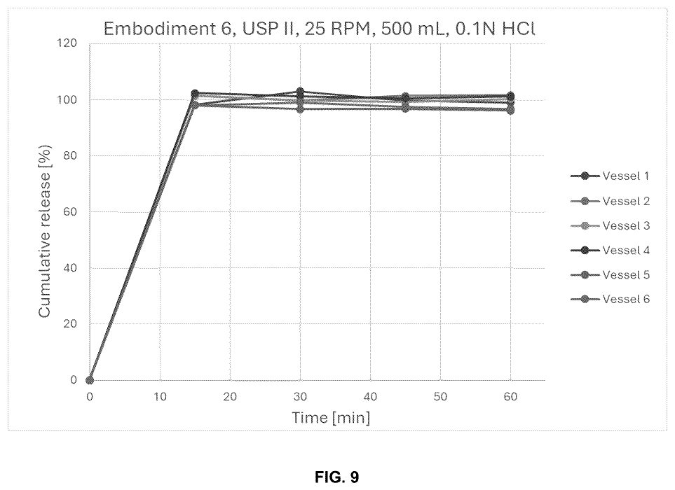

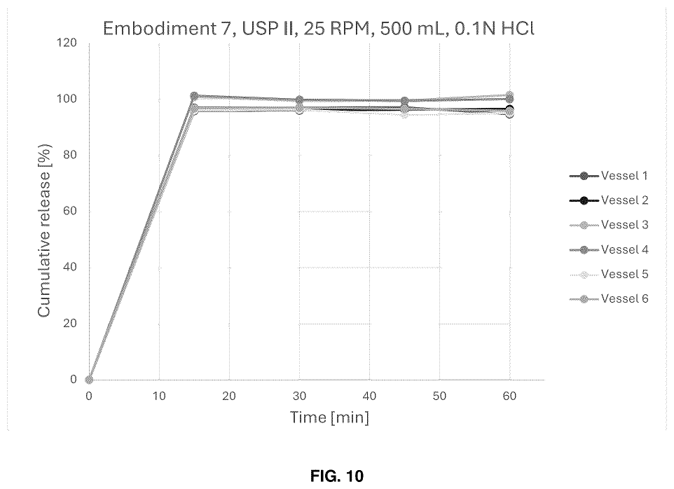

Embodiments 5-7 were prepared as described in Example 8. Dissolution testing was conducted on freshly manufactured Embodiments 5, 6 and 7 using the USP II (Paddle) Dissolution Apparatus by placing the sample of the composition in 500 mL of 0.1 N HCl and using a paddle stir rate of 25 rpm. FIGS. 8-10 show the dissolution profile of Embodiments 5-7, respectively. Each sample of the embodiments had a consistent dissolution rate of greater than 85% dissolution within 15 minutes. This data show that the compositions described herein are rapidly dissolving.

### Example 11: Comparison of LABRASOL® and LABRASOL®ALF

LABRASOL® ALF is aldehyde free. In order to assess if there is any difference with respect to impact on stability between LABRASOL® ALF and LABRASOL® (which contains aldehydes), a chemical compatibility study of both grades of LABRASOL® with Lenalidomide and Temozolomide has been performed.

Temozolomide and Lenalidomide quaternary formulations were prepared involving two variations: one with LABRASOL® ALF and the other with standard LABRASOL®. The quaternary formulations for Temozolomide and Lenalidomide are presented in Table 19 and Table 20, retrospectively.

The quaternary samples were prepared under atmospheric conditions. The API was weighed to the correct amount. The other components Silicon Dioxide (as Aerosil 200P™), MIGLYOL® 812N, and either LABRASOL® or LABRASOL® ALF, depending on the formulation, were also weighed to their correct amount. These components were added sequentially to the API in the following order: Silicon Dioxide (as Aerosil 200P™), MIGLYOL® 812N and LABRASOL®/LABRASOL® ALE and mixed until homogeneous.

Each sample was filled into clear 40 mL EPA vials, with each vial containing 20 mL of the quaternary formulation. The vials were then sealed with a plastic cap, that included a PTFE/silicone seal, and wrapped in foil. The samples were stored at 25° C./60% RH and 4000/75% RH. After 28 days, the samples were analyzed for their related substances content to evaluate the impact of the LABRASOL® variants on the chemical stability of Temozolomide and Lenalidomide. The results are presented in Tables 21-24.

TABLE 19
Sample compositions evaluated in Temozolomide
Chemical Compatibility Study

| Material | Sample 1 | | Sample 2 | |
|---|---|---|---|---|
| Temozolomide | 40.0 | mg/mL | 40.0 | mg/mL |
| MIGLYOL ® 812N | 865.0 | mg/mL | 865.0 | mg/mL |
| Silicon Dioxide (Aerosil 200P ™) | 15.0 | mg/mL | 15.0 | mg/mL |

| | | | |
|---|---|---|---|
| LABRASOL ® ALF | 50.0 | mg/mL | N/A |

| | | | |
|---|---|---|---|
| LABRASOL | N/A | 50.0 | mg/mL |

TABLE 20
Sample compositions evaluated in Lenalidomide
Chemical Compatibility Study

| Material | Sample 3 | | Sample 4 | |
|---|---|---|---|---|
| Lenalidomide | 5.0 | mg/mL | 5.0 | mg/mL |
| MIGLYOL ® 812N | 900.0 | mg/mL | 900.0 | mg/mL |
| Silicon Dioxide (Aerosil 200P ™) | 15.0 | mg/mL | 15.0 | mg/mL |

| | | | |
|---|---|---|---|
| LABRASOL ® ALF | 50.0 | mg/mL | N/A |

| | | | |
|---|---|---|---|
| LABRASOL ® | N/A | 50.0 | mg/mL |

TABLE 21
Stability of Temozolomide Sample 1 (containing LABRASOL ® ALF)
and Temozolomide Sample 2 (containing LABRASOL ®) at
25° C./60% RH (28 Days)-presence of impurities

28 Days (25° C./60% RH)

| Sample | SLU* | Tot** |
|---|---|---|
| 1 | n.d*** | n.d*** |
| 2 | n.d*** | n.d*** |

*Single largest unknown impurity
**Total impurities
***Not detected

TABLE 22
Stability of Temozolomide Sample 1 (containing LABRASOL ® ALF)
and Temozolomide Sample 2 (containing LABRASOL ®) at
40° C./75% RH (28 Days)-presence of impurities

28 Days (40° C./75% RH)

| Sample | SLU* | Tot** |
|---|---|---|
| 1 | 0.1 | 0.1 |
| 2 | 0.1 | 0.1 |

*Single largest unknown impurity
**Total impurities

TABLE 23
Stability of Lenalidomide Sample 3 (containing LABRASOL ® ALF)
and Lenalidomide Sample 4 (containing LABRASOL ®) at
25° C./60% RH (28 Days)-presence of impurities

28 Days (25° C./60% RH)

| Sample | SLU* | Tot** |
|---|---|---|
| 3 | 0.06 | 0.06 |
| 4 | 0.21 | 0.32 |

*Single largest unknown impurity
**Total impurities

TABLE 24
Stability of Lenalidomide Sample 3 (containing LABRASOL ® ALF)
and Lenalidomide Sample 4 (containing LABRASOL ®) at
40° C./75% RH (28 Days)-presence of impurities

28 Days (40° C./75% RH)

| Sample | SLU* | Tot** |
|---|---|---|
| 3 | 0.13 | 0.32 |
| 4 | 0.38 | 0.51 |

*Single largest unknown impurity
**Total impurities

The data shown above illustrate that the temozolomide samples manufactured with either LABRASOL® ALF or LABRASOL® were stable after 28 days storage at both 25° C./60% RH and 40° C./75% RH. The lenalidomide formulation manufactured with LABRASOL® ALF was stable after 28 days storage at both 25° C./60% RH and 40° C./75% RH. However, the lenalidomide formulation manufactured with LABRASOL® was stable after 28 days storage at 25° C./60% RH but was not stable at 40° C./75% RH.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A composition comprising:
about 0.25 to about 100 mg/mL active pharmaceutical ingredient (API);
about 400 to about 960 mg/mL medium chain triglycerides (MCT);
about 1 to about 20 mg/mL silicon dioxide; and
about 20 to about 70 mg/mL surfactant;
wherein the API is temozolomide or lenalidomide,
wherein the composition is substantially free of water and anti-microbial preservatives, and
wherein the composition is an oral suspension.

2. The composition of claim 1, wherein the API is temozolomide, and an amount of temozolomide is between about 5 to about 100 mg/mL.

3. The composition of claim 1, wherein the API is lenalidomide, and an amount of lenalidomide is between about 0.25 to about 50 mg/mL.

4. The composition of claim 1, wherein the API is micronized.

5. The composition of claim 1, wherein the MCT is present in an amount between about 850 to about 980 mg/mL.

6. The composition of claim 1, wherein the silicon dioxide is present in an amount of about 12.1 mg/mL to about 12.5 mg/mL.

7. The composition of claim 1, wherein the surfactant is present in an amount of between about 40 to about 50 mg/mL.

8. The composition of claim 7, wherein the surfactant is selected from the group consisting of aldehyde-free caprylocaproyl poloxyl-8 glycerides, caprylocaproyl poloxyl-8 glycerides, polysorbate 80, polysorbate 20, sorbitan monolaurate, and vitamin E polyethylene glycol succinate.

9. The composition of claim 1, wherein the composition further comprises a sweetener and/or a flavoring agent.

10. The composition of claim 9, herein the sweetener is present in an amount of 0.1 to about 3 mg/mL, and wherein the sweetener is selected from the group consisting of sucralose, aspartame, acesulfame potassium, saccharin, saccharin sodium, and neotame.

11. The composition of claim 9, wherein the flavoring agent is present in an amount of between about 0.1 to about 5 mg/mL, and wherein the flavoring agent is selected from the group consisting of grape flavoring, cola flavoring, cherry flavoring, berry flavoring, citrus flavoring, raspberry flavoring, mint flavoring, orange flavoring, caramel flavoring, and tutti frutti flavoring.

12. The composition of claim 1, further comprising an antioxidant.

13. The composition of claim 12, wherein the antioxidant is present in an amount of between about 0.05 to about 2 mg/mL, and wherein the antioxidant is selected from the group consisting of butylated hydroxytoluene (BHT), butylated hydroxyanisole (BHA), ascorbyl palmitate, and propyl gallate.

14. The composition of claim 1, wherein the composition is in the form of a multidose formulation.

15. The composition of claim 1, wherein the composition is free or substantially free of an organic solvent.

16. The composition of claim 1, wherein the composition is free or substantially free of an acid.

17. The composition of claim 1, comprising:
about 5 to about 100 mg/mL temozolomide;
about 400 to about 960 mg/mL MCT;
about 1 to about 20 mg/mL silicon dioxide;
about 0.1 to about 3 mg/mL sweetener;
about 20 to about 70 mg/mL surfactant; and
about 0.1 to about 5 mg/mL flavoring agent.

18. The composition of claim 1, comprising:
about 5 to about 100 mg/mL temozolomide;
about 400 to about 960 mg/mL MCT;
about 1 to about 20 mg/mL silicon dioxide;
about 0.1 to about 3 mg/mL sweetener;
about 20 to about 70 mg/mL surfactant;
about 0.1 to about 5 mg/mL flavoring agent; and
about 0.05 to about 2 mg/mL antioxidant.

19. The composition of claim 17, comprising:
about 40 mg/mL temozolomide;
about 868.7 mg/mL MCT;
about 12.1 mg/mL silicon dioxide;
about 1.9 mg/mL sucralose;
about 2.9 mg/mL grape flavoring agent; and
about 44.4 mg/mL caprylocaproyl poloxyl-8 glycerides.

20. The composition of claim 18, comprising:
about 40 mg/mL temozolomide;
about 868.5 mg/ml MCT;
about 12.1 mg/mL silicon dioxide;
about 1.9 mg/mL sucralose;
about 2.9 mg/mL grape flavoring agent;
about 44.4 mg/mL caprylocaproyl poloxyl-8 glycerides; and
about 0.2 mg/mL BHT.

21. The composition of claim 18, comprising:
about 40 mg/mL temozolomide;
about 868.4 mg/mL MCT;
about 12.1 mg/mL silicon dioxide;
about 1.9 mg/mL sucralose;
about 2.9 mg/ml grape flavoring agent;
about 44.4 mg/mL caprylocaproyl poloxyl-8 glycerides; and
about 0.3 mg/mL BHT.

22. The composition of claim 1, comprising:
about 0.25 to about 50 mg/mL lenalidomide;
about 450 to about 980 mg/mL MCT;
about 1 to about 20 mg/mL silicon dioxide;
about 0.1 to about 3 mg/mL sweetener;
about 20 to about 70 mg/mL surfactant; and
about 0.1 to about 5 mg/mL flavoring agent.

23. The composition of claim 22, comprising:
about 5 mg/mL lenalidomide;
about 902.5 mg/mL MCT;
about 12.5 mg/mL silicon dioxide;
about 2.0 mg/mL sucralose;
about 3.0 mg/ml grape flavoring agent; and
about 45.0 mg/mL aldehyde-free caprylocaproyl poloxyl-8 glycerides.

24. The composition of claim 22, comprising:
about 5 mg/mL lenalidomide;
about 902.0 mg/mL MCT;
about 12.5 mg/mL silicon dioxide;
about 2.0 mg/mL sucralose;
about 3.0 mg/ml grape flavoring agent; and
about 45.0 mg/mL aldehyde-free caprylocaproyl poloxyl-8 glycerides; and
about 0.5 mg/mL BHT

25. A method for treating cancer, the method comprising:
administering to a subject in need thereof the composition of claim 1.

26.-40. (canceled)
